(19) United States
(12) Patent Application Publication
(10) Pub. No.: US 2021/0128680 A1
(43) Pub. Date: May 6, 2021

(54) METHOD FOR TREATING CARDIOPULMONARY DISORDER

(71) Applicants: Chang Gung University, Taoyuan City (TW); Chang Gung Memorial Hospital, Linkou, Taoyuan City (TW)

(72) Inventors: Ying-Ju LAI, Taoyuan City (TW); Pao-Hsien CHU, Taoyuan City (TW); Whei-Yang Winston KAO, Cincinnati, OH (US)

(73) Assignees: Chang Gung University, Taoyuan City (TW); Chang Gung Memorial Hospital, Linkou, Taoyuan City (TW)

(21) Appl. No.: 16/669,495

(22) Filed: Oct. 30, 2019

(51) Int. Cl. A61K 38/16; A61P 9/12; A61K 31/5585; A61K 31/5578; A61K 31/505; A61K 31/519; A61K 31/4985; A61K 31/4422; A61K 31/554; A61K 31/506; A61K 31/366

(52) U.S. Cl. CPC A61K 38/16; A61P 9/12; A61K 31/5585; A61K 31/5578; A61K 31/505; A61K 31/366; A61K 31/4985; A61K 31/4422; A61K 31/554; A61K 31/506; A61K 31/519

(57) ABSTRACT

Provided herein are synthetic peptides and methods for treating cardiorespiratory diseases, particularly cardiopulmonary disorder (CPD) by use of the synthetic peptides. The present synthetic peptide has the amino acid sequence of X₁EX₂LRVANEVTLN (SEQ ID NO: 1), in which X₁ is tyrosine (Y) or phenylalanine (F), and X₂ is alanine (A) or cysteine (C). In preferred embodiments, an effective amount of the present synthetic peptide is administered to a subject suffering from CPD to ameliorate or alleviate symptoms associated therewith.

### BACKGROUND OF THE INVENTION

### 1. Field of the Invention

The present disclosure relates to the treatment of a cardiopulmonary disorder (CPD), particularly, to the treatment of pulmonary hypertension (PH).

### 2. Description of Related Art

The cardiopulmonary vascular system is the key determinant of prognosis on cardiorespiratory diseases, particularly cardiopulmonary disorder (CPD), which encompasses a range of serious disorders that affect the heart (“cardio-”) and/or the lungs (“-pulmonary”), such as coronary artery disease (CAD), atherosclerosis, peripheral artery disease, and pulmonary hypertension (PH) (e.g., pulmonary arterial hypertension (PAH)).

PAH is a rare, chronic, progressive disease characterized by elevated blood pressure in the pulmonary arteries. This elevated pressure ultimately results in right ventricular failure and death. Patients are normally severely affected, with a life expectancy of only a few years after the first symptom occurred.

PAH is often asymptomatic in the beginning and is typically diagnosed late in its course. The first symptom of PAH is usually shortness of breath with everyday activities, such as climbing stairs. Fatigue, dizziness and fainting can also be symptoms, as can irregular heartbeat (palpitations or strong, throbbing sensation), racing pulse, progressive shortness of breath during exercise or activity, and difficulty breathing at rest. Swelling of the ankles, abdomen or legs, bluish lips and skin and chest pain may occur as strain on the heart increases. In more advanced stages of the disease, even minimal activity will produce some of the symptoms. Eventually, it may become difficult to carry out any activities as the disease worsens.

Despite improvements in the diagnosis and management of PAH with the introduction of targeted medical therapies leading to improved survival, the disease continues to have a poor long-term prognosis, while mortality and hospitalization rates continue to increase.

In view of the foregoing, there exists in the related art a need of an improved treatment of PAH.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method of treating a cardiopulmonary disorder (CPD) in a subject. The method includes the step of administering to the subject an effective amount of a synthetic peptide to ameliorate or alleviate the symptoms associated with the CPD, in which the synthetic peptide has an amino acid sequence of X₁EX₂LRVANEVTLN (SEQ ID NO: 1), where X₁ is tyrosine (Y) or phenylalanine (F), and X₂ is alanine (A) or cysteine (C).

According to some preferred embodiments of the present disclosure, in the synthetic peptide of SEQ ID NO: 1, X₁ is tyrosine (Y) and X₂ is alanine (A), thus gives rise to the synthetic peptide of SEQ ID NO: 2.

According to other embodiments of the present disclosure, in the synthetic peptide of SEQ ID NO:1, X₁ is tyrosine (Y) and X₂ is cysteine (C), thus gives rise to the synthetic peptide of SEQ ID NO: 3.

According to further embodiments of the present disclosure, in the synthetic peptide of SEQ ID NO:1, X₁ is phenylalanine (F) and X₂ is alanine (A), thus gives rise to the synthetic peptide of SEQ ID NO: 4.

Examples of the CPD that may be treated by the present method include, but are not limited to, coronary artery disease, atherosclerosis, peripheral artery disease, and pulmonary hypertension (PH).

According to further embodiments of the present disclosure, the PH is pulmonary arterial hypertension (PAH).

Alternatively, or optionally, the present method further includes the step of administering to the subject an agent selected from the group consisting of vasodilator, endothelin receptor antagonist, phosphodiesterase 5 (PDE5) inhibitor, calcium channel blocker, soluble guanylate cyclase (SGC) stimulator, anticoagulant, digoxin, diuretics, and oxygen, to ameliorate or alleviate the symptoms associated with the CPD, such as PH.

Examples of the vasodilator suitable for use in the present method include, but are not limited to, epoprostenol, iloprost, and treprostinil. Examples of the endothelin receptor antagonist suitable for use in the present method include, but are not limited to, bosentan, macitentan, and ambrisentan. Examples of the PDE5 inhibitor include sildenafil and tadalafil. Examples of the calcium channel blocker include amlodipine, diltiazem, and nifedipine. Example of the SGC stimulator is riociguat, and example of the anticoagulant is warfarin.

According to embodiments of the present disclosure, the peptide is administered to the subject in the amount of 10 ng/day to 50 mg/day. Preferably, the peptide is administered to the subject in the amount of 500 ng/day to 30 mg/day.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIGS. 1A to 1C. Loss lumican is a marker for PAH. 1A. Western blot analysis of HIF-1α, and lumican relative to GAPDH in the human lung tissue. Lower panel: Relative expression values obtained by densitometry of HIF-1α, and lumican protein normalized to glyceraldehyde-3-phosphate dehydrogenase (GAPDH) (n=4 for each group). 1B. Western blot analysis of lumican in the organs of monocrotaline (MCT)-induced PH rats and control animals. 1C. Western blot analysis of lumican in the organs of hypoxia-induced PH mice (Hypo) and the control animals. Western blot analysis was performed with anti-HIF-1α and lumican antibody at a molecular weight about 130 kDa and 42 kDa. Quantification of proteins is shown in the bar graph. The bars represent the mean±SEM of four samples in each group. *P<0.05 compared with the CON group.

FIG. 2. Lumican expression is specific to pulmonary arteries smooth muscle cells. (A) immunohistochemical photographs showed lumican expression in the proximal (scale bar: 100, and 25 μm) and distal parts of PAs (scale bar: 50 μm) in human lung tissue. Lumican in the smooth muscle layer of PAs was identified by lumican staining (brown staining). (B) Lumican immunohistochemistry staining in pulmonary arteries (scale bar: 50 μm) in diameter in control and MCT rat lung tissue. (C) Lumican immunohistochemistry staining in pulmonary arteries (scale bar: 50 μm) in diameter in normoxia and hypoxia mouse lung tissue. Scale bar=100 μm. (The gradient color from black and gray arrow indicated that the expression lumican was loss from the proximal to the distal lung tissue.)

FIG. 3. Lumican null mice are promoted the development of hypoxic PAH. (A) Western blot analysis of lumican in lumican null and WT mice lung tissues. Relative expression values obtained by densitometry of lumican protein normalized to GAPDH (n=3 per group). The bars represent the mean±SEM of four samples in each group. ***P<0.001 compared with the WT in normoxic condition. (B) The ratio of RV to LV plus septum weight (RV/LV+S) in 4 different groups is shown in WT mice as well as lumican null and lumican null and littermates at 4 weeks under hypoxic conditions of 10% oxygen. The bars represent the mean±SEM of four samples in each group. P*<0.05, and ***P<0.001 compared with the WT in normoxic condition, ^{###}P<0.001 compared with the WT in hypoxic condition. (for all groups, n=15) (C) Medial wall thickness (MWT) of small PAs (25-50 μm) identified by α-SM-actin staining. The degree of MWT was compared among 4 groups. Each value (mean±SE [n=10]) is expressed. **P<0.01, ***p<0.001 versus the WT in normoxic condition, or ###P<0.001 compared with the WT in hypoxic condition, one-way ANOVA, Bonferroni posttest.

FIG. 4. Effect of lumikine on the hypoxia induced AKT phosphorylation and p-SMAD2 in the cultured human PASMCs. (A). human PASMCs were treated with an in vitro hypoxic (1% oxygen) model for 24 hours with or without lumikine compared with PASMCs in normoxic condition. Western blot shows protein expression in whole cell extraction. Relative expression values obtained by densitometry of lumican (B) and HIF-1α (C), pSmad2 (D), pAKT (E) and Smad4 (F). The bars represent the mean±SEM of three samples in each group. ***P<0.001 compared with the PASMCs in normoxic condition. ##P<0.01 compared with the PASMCs in hypoxic condition, one-way ANOVA, Bonferroni posttest.

FIG. 5. Effects of the lumikin on the proliferation of MCT PASMC via non-canonical p-AKT signaling. (A). Lumican was detected as 42-kDa bands, and the protein levels were decreased in MCT PASMCs compared with CON PASMCs. The bars represent the mean±SEM of three samples in each group. *P<0.05 compared with the CON PASMCs. (B). The lumikine inhibited significantly the proliferation of MCT PASMCs. PASMCs were exposed to the PKA-AKT inhibitor (0.1 l, and 10 μM) and incubated with or without lumikine (100 nM). Lumikine inhibited the proliferation of MCT PASMCs, which was reversed by a PKA-AKT (H89) inhibitors. However, lumikine did not affect the proliferation control (CON) PASMCs. Each value (mean±SE [n=4]) is expressed. ***p<0.001 versus 10% FBS only group in MCT PASMCs, and shown decreased, #p<0.01, ###p<0.001 versus 10% FBS only group in MCT PASMCs, shown increased, one-way ANOVA, Bonferroni posttest.

FIG. 6. The effect of lumikin on the right ventricular hypertrophy and the degree of muscularization. (A) The ratio of RV to LV plus septum (RV/LV+S) weight. Lumikine treatment led to a reduction in the RV/LV+S ratio compared with normoxic mice group (n=14) (n=8) or chronically hypoxic mice group (n=15) (n=15). ***P<0.001 versus normoxic mice group; #P<0.05 compared with hypoxic mice group. (B). The medial wall thickness (MWT) of pulmonary arteries (25-100 μm) was identify by α-SMA staining (brown). The MWT of pulmonary arteries with a diameter between 25 to 100 μm in normoxic mice group or chronically hypoxic mice and with or without lumikine at a dosage of 3 ng/kg/day on days 8-28. ***P<0.001 compared with the normoxic mice group without lumikine. ##P<0.01 compared with hypoxic mice group. The data are presented as the mean±SE; ANOVA with Bonferroni's post hoc test.

### DESCRIPTION

The detailed description provided below in connection with the appended drawings is intended as a description of the present examples and is not intended to represent the only forms in which the present example may be constructed or utilized. The description sets forth the functions of the example and the amino acid sequence of steps for constructing and operating the example. However, the same or equivalent functions and sequences may be accomplished by different examples.

### 1. Definitions

For convenience, certain terms employed in the specification, examples and appended claims are collected here. Unless otherwise defined herein, scientific and technical terminologies employed in the present disclosure shall have the meanings that are commonly understood and used by one of ordinary skill in the art.

As used herein, the term “peptide” denotes a polymer of amino acid residues. By the term “synthetic peptide” as used herein, it is meant a peptide which does not comprise an entire naturally occurring protein molecule. The peptide is “synthetic” in that it may be produced by human intervention using such techniques as chemical synthesis, recombinant genetic techniques, or fragmentation of whole protein or the like. Throughout the present disclosure, the positions of any specified amino acid residues within a peptide are numbered starting from the N terminus of the peptide. When amino acids are not designated as either D- or L-amino acids, the amino acid is either an L-amino acid or could be either a D- or L-amino acid, unless the context requires a particular isomer. Further, the notation used herein for the polypeptide amino acid residues are those abbreviations commonly used in the art.

As discussed herein, minor variations in the amino acid sequences of proteins/peptides are contemplated as being encompassed by the presently disclosed and claimed inventive concept(s), providing that the variations in the amino acid sequence maintain at least 90% identical, such as at least 70%, 71%, 72%, 73%, 75%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99% identical. The present synthetic peptide may be modified specifically to alter a feature of the peptide unrelated to its physiological activity. For example, certain amino acids can be changed and/or deleted without affecting the physiological activity of the peptide in this study (i.e., its ability to treat pulmonary hypertenion). In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains. In one example, one amino acid residue (e.g., aspartate, valine or phenylalanine) of the present synthetic peptide is conservatively replaced by non-polar amino acid residue (e.g., by alanine). In other examples, one amino acid residue of the present synthetic peptide is conservatively replaced by its D-form amino acid residue, for example, L-form arginine and L-form phenylalanine are respectively replaced by the corresponding D-form residues.

The term “treatment” as used herein are intended to mean obtaining a desired pharmacological and/or physiologic effect, e.g., delaying or inhibiting pulmonary arterial hypertension (PAH) induced proliferation of pulmonary smooth muscle cells. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., development of PAH) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, intravascular delivery (e.g., injection or infusion), oral, enteral, pulmonary (e.g., inhalation), nasal, topical (including transdermal, buccal and sublingual), intraperitoneal, brain delivery (e.g., intracerebroventricular, and intracerebral), or parenteral (e.g., subcutaneous, intramuscular, intravenous, and intradermal), transmucosal administration or administration via an implant, or other delivery routes known in the art. In some embodiments, the synthetic peptide of the present disclosure and/or its analogues are formulated suitable for inhalation. In further embodiments, the synthetic peptide of the present disclosure and/or its analogues are formulated into powders for mixed with suitable carrier (e.g., buffer solution) before use, such as intraveneous injection.

The term “an effective amount” as used herein refers to an amount effective, at dosages, and for periods of time necessary, to achieve the desired result with respect to the treatment of a disease. For example, in the treatment of CPD (e.g., PH, PAH and the like), an agent (i.e., a synthetic peptide, or a nucleic acid encoding a therapeutic peptide) which decrease, prevents, delays or suppresses or arrests any symptoms of the CPD would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

The term “subject” or “patient” is used interchangeably herein and is intended to mean a mammal including the human species that is treatable by the synthetic peptide and/or method of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In an exemplary embodiment, the patient is a human.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

### 2. Method of Treating Cardiopulmonary Disorder (CPD)

The invention disclosed herein focuses on the treatment of a cardiopulmonary disorder, such as pulmonary hypertension (PH), particularly, pulmonary arterial hypertension (PAH). To this end, synthetic peptides having the ability of reversing PAH induced reduction of lumican expressoin, as well as suppressing the PAH induced proliferation of pulmonary artery smooth muscle cells (PASMCs), are provided by the present disclosure. As could be appreciated, an expression system, such as a nucleic acid encoding the above-mentioned peptides, also falls within the scope of the present disclosure.

Accordingly, the present method comprises the step of administering to a subject suffering from a pulmonary disease an effective amount of a synthetic peptide of the present disclosure, to ameliorate or alleviate the symptoms associated with the pulmonary disease, wherein the synthetic peptide has an amino acid sequence of X₁EX₂LRVANEVTLN (SEQ ID NO: 1), in which X₁ is tyrosine (Y) or phenylalanine (F), and X₂ is alanine (A) or cysteine (C).

According to some embodiments of the present disclosure, in the synthetic peptide of SEQ ID NO:1, X₁ is tyrosine (Y) and X₂ is alanine (A), thus gives rise to the synthetic peptide of SEQ ID NO: 2, which is also term “lumikine” in the present disclosure. According to other embodiments of the present disclosure, in the synthetic peptide of SEQ ID NO:1, X₁ is tyrosine (T) and X₂ is cysteine (C), thus gives rise to the synthetic peptide of SEQ ID NO: 3. According to further embodiments of the present disclosure, in the synthetic peptide of SEQ ID NO:1, X₁ is phenylalanine (F) and X₂ is alanine (A), thus gives rise to the synthetic peptide of SEQ ID NO: 4.

The present synthetic peptide may be synthesized in accordance with any standard peptide synthesis protocol in the art. In one embodiment, the present synthetic peptides were synthesized by use of a solid-phase peptide synthesizer (ABI433A peptide synthesizer, Applied Biosystems Inc., Life Technologies Corp., Foster City, Calif., USA) in accordance with the manufacturer's protocols.

Alternatively, the present synthetic peptides may be prepared using recombinant technology. For example, one can clone a nucleic acid encoding the present peptide in an expression vector, in which the nucleic acid is operably linked to a regulatory sequence suitable for expressing the present peptide in a host cell. One can then introduce the vector into a suitable host cell to express the peptide. The expressed recombinant polypeptide can be purified from the host cell by methods such as ammonium sulfate precipitation and fractionation column chromatography. A peptide thus prepared can be tested for its activity according to the method described in the examples below.

The above-mentioned nucleic acids or polynucleotide can be delivered by the use of polymeric, biodegradable microparticle or microcapsule delivery devices known in the art. Another way to achieve uptake of the nucleic acid in a host is using liposomes, prepared by standard methods. The polynucleotide can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Alternatively, tissue specific targeting can be achieved by the use of tissue-specific transcriptional regulatory elements that are known in the art. Delivery of “naked DNA” (i.e., without a delivery vehicle) to an intramuscular, intradermal, or subcutaneous site is another means to achieve in vivo expression.

The present synthetic peptide may be modified at its N-terminus or C-terminus. Examples of N-terminal modifications include, but are not limited to, N-glycated, N-alkylated, and N-acetylated amino acid. A terminal modification can include a pegylation. An example of C-terminal modification is a C-terminal amidated amino acid. Alternatively, one or more peptide bond may be replaced by a non-peptidyl linkage, the individual amino acid moieties may be modified through treatment with agents capable of reacting with selected side chains or terminal residues.

Various functional groups may also be added at various points of the synthetic peptide that are susceptible to chemical modification. Functional groups may be added to the termini of the peptide. In some embodiments, the function groups improve the activity of the peptide with regard to one or more characteristics, such as improving the stability, efficacy, or selectivity of the synthetic peptide; improving the penetration of the synthetic peptide across cellular membranes and/or tissue barrier; improving tissue localization; reducing toxicity or clearance; and improving resistance to expulsion by cellular pump and the like. Non-limited examples of suitable functional groups are those that facilitate transport of a peptide attached thereto into a cell, for example, by reducing the hydrophilicity and increasing the lipophilicity of the peptide, these functional groups may optionally and preferably be cleaved in vivo, either by hydrolysis or enzymatically, inside the cell. Hydroxy protecting groups include esters, carbonates and carbamate protecting groups. Amine protecting groups include alkoxy and aryloxy carbonyl groups. Carboxylic acid protecting groups include aliphatic, benzylic and aryl esters. In some optional embodiments, the carboxylic acid group in the side chain of the aspartic acid (D) of the present synthetic peptide is protected, preferably, by a methyl, ethyl, benzyl, or substituted benzyl ester.

A “peptidomimetic organic moiety” can optionally be substituted for amino acid residues in the present synthetic peptide both as conservative and as non-conservative substitutions. The peptidomimetic organic moieties optionally and preferably have steric, electronic or configuration properties similar to the replaced amino acid and such peptidomimetics are used to replace amino acids in the essential positions, and are considered conservative substitutions. Peptidomimetics may optionally be used to inhibit degradation of peptides by enzymatic or other degradative processes. The peptidomimetics can optionally and preferably be produced by organic synthetic techniques. Non-limiting examples of suitable peptidomimetics include isosteres of amide bonds, 3-amino-2-propenidone-6-carboxylic acid, hydroxyl-1,2,3,4-tetrahydro-isoquinoline-3-carboxylate, 1,2,3,4-tetrahydro-isoquinoline-3-carboxylate, and histidine isoquinolone carboxylic acid.

Any part of the synthetic peptide may optionally be chemically modified, such as by the addition of functional groups. The modification may optionally be performed during the synthesis of the present peptide. Non-limiting exemplary types of the modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxy groups to an amino group of a sugar. Acetal and ketal bonds can also optionally be formed between amino acids and carbon hydrates.

According to embodiments of the present disclosure, the synthetic peptide of the present disclosure is administered to the subject via inhalation. Depending on the type and severity of the disease, about 10 ng/day to about 50 mg/day of the present synthetic peptide is administered to the patient, such as 10, 20, 30, 40, 50, 60, 70, 80, 90, 100, 200, 300, 400, 500, 600, 700, 800, 900 ng/day, and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/day. A typical daily dosage might range from about 500 ng/day to about 30 mg/day, such as 500, 600, 700, 800, 900 ng/day, and 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 mg/day; preferably about 5 mg/day. The doses utilized for the purposes described above will generally be administered one to several, e.g., four, six, eight or even more, times per day.

Optionally, the present method may further include administering to the subject an effective amount of an agent selected from the group consisting of vasodilator (e.g., epoprostenol, iloprost, and treprostinil), endothelin receptor antagonist (e.g., bosentan, macitentan, and ambrisentan), phosphodiesterase 5 (PDE5) inhibitor (e.g., sildenafil and tadalafil), calcium channel blocker (e.g., amlodipine, diltiazem, and nifedipine), soluble guanylate cyclase (SGC) stimulator (e.g., riociguat), anticoagulant (e.g., warfarin), digoxin, diuretics, and oxygen to treat pulmonary artery hypertension. In some embodiments, oxygen is administered to the subject in addition to the present synthetic peptide (e.g., SEQ ID NO: 2). In other embodiments, the vasodilator (e.g., epoprostenol, iloprost, and treprostinil) is administered to the subject in addition to the present synthetic peptide (e.g., SEQ ID NO: 2). In still further embodiments, the SGC stimulator (e.g., riociguat) is administered to the subject in addition to the present synthetic peptide (e.g., SEQ ID NO: 2).

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention fully.

### EXAMPLES

Materials and Methods

Patient Characteristics and Measurements.

Human lung tissue was obtained from 4 non-PAH and 4 idiopathic pulmonary arterial hypertension (IPAH) patients undergoing surgery at National Taiwan University Hospital, see Table 1 for details on patient information. Lung tissue was snap-frozen directly after explanation for protein extraction. The study protocol for tissue donation was approved by the Human Research Ethics Committee at National Taiwan University Hospital (Institutional Review Board 201409069RINA) and Chang Gung Memorial Hospital (Chang Gung Medical Foundation Institutional Review Board 104-0287B), and the study was conducted in accordance with the principles of the Declaration of Helsinki. Written informed consent was obtained from each patient.

TABLE 1
Characteristics of Patients and non-PAH
A. non-PAH lung

| Pa- | | | |
|---|---|---|---|
| tient | Age- | | |
| # | Gender | Diagnosis | Assay Method |
| 1 | 53-M | Lung Cancer | WB, IHC |
| 2 | 63-F | COPD | WB |
| 3 | 69-F | Lung cancer | WB |
| 4 | 74-M | Lung cancer | WB |

B. IPAH patients

| Pa- | | | | | PAH | |
|---|---|---|---|---|---|---|
| tient | Age- | PAP | PVR | 6 | Medi- | Assay |
| # | Gender | (s/d/m) | (WU) | MW | cations | Method |
| 1 | 54-F | 92/37/55 | 13.76 | 238 | Bosentan | WB |
| | | | | | Viagra | |
| | | | | | Lasix | |
| 2 | 27-F | 81/40/55 | 19.8 | 93 | Aldactone | WB |
| | | | | | Opsumit FC | |
| | | | | | Lanoxin | |
| 3 | 55-M | 102/45/69 | 23.9 | 360 | Remodulin | WB |
| | | | | | Macitentan | IHC |
| 4 | 52-M | 75/34/50 | 11.23 | 410 | Sildenafil | WB |
| | | | | | cordarone | |

COPD, Chronic Obstructive Pulmonary Disease
IPAH, idiopathic pulmonary arterial hypertension
PAP, pulmonary artery pressure (mmHg), s: systolic, d: diastolic, m: mean
PVR, pulmonary vascular resistance (dynes/sec.com-5)
6 MW, distance (m), walked in 6 minutes
IHC, Immunohistochemistry
WB, protein expression assayed by western immunoblot

Pulmonary Hypertension (PH) Rat Model

Adult male Sprague-Dawley rats (200-250 g in body weight) were randomized assigned into two groups for treatment of 28 days after s.c. injection of saline or 60 mg/kg monocrotaline (MCT) (Sigma-Aldrich) to induce pulmonary hypertension (PH). Rats were examined after 3 weeks of treatment (on day 28). Hypoxic pulmonary vascular remodeling was induced by exposure of mice (8-week-old C57BL/6) to chronic hypoxia (10% 02) in a ventilated chamber, as described previously (Li H H et al. Am J Physiol Lung Cell Mol Physiol. 2017: ajplung 00245 2017). The chronic effects of lumikne were assessed in mice exposed to hypoxia for 28 days. Briefly, animals were kept in hypoxic conditions to develop pulmonary hypertension. After 7 days, animals were randomized to receive either I.P. (1.5 μg/kg/day) or saline. All animal experimental protocols were reviewed and approved by the Chang Gung University IACUC. This study was carried out in strict accordance with the recommendations in the Guide for the Care and Use of Laboratory Animals of the National Institutes of Health. Housing and maintenance was provided by Chang Gung University, and all animals were fed a standard chow diet with free access to water.

Generation and Maintenance of Lumican-Deficient (Lumican-Null) Mice

Male C57BL/6 mice (either wild-type or lumican deficient) aged 2 months, were obtained from Chang Gung University Laboratory Animal Center. Lumican-null mice were generated by targeted gene disruption as previously described (Saika S et al. J Biol Chem. 2000, 275(4):2607-12; and Kao W W et al., Exp Eye Res. 2006, 82(1):3-4.). All procedures for handling mice conformed to Association of Research for Vision and Ophthalmology guidelines. Statements for the use of animals in research were approved by the Chang Gung University IACUC.

Assessment of RV Hypertrophy.

After sacrifice, the RV wall was separated from the LV wall and ventricular septum. Wet weight of the RV, free LV wall, and ventricular septum was determined and weighted. RV hypertrophy was expressed as the ratio of weight of the RV wall and that of the free LV wall and ventricular septum (LV+S).

Paraffin Embedding and Microscopy.

Fixation was performed by immersion of the lungs in a 10% paraformaldehyde solution. For paraffin embedding, the entire lungs were dissected in tissue blocks from all lobes. Sectioning at 5 μm was performed from all paraffin-embedded blocks. To assess the type of remodeling of muscular pulmonary arteries, the percentage of MWT was utilized to characterize vascular medial hypertrophy by α-SM-actin staining. Under 400× microscopic examination, MWT was defined as the distance between the internal and external elastic laminae using “ImageJ” software downloaded from the open source available from the internet. For vascular sections, the diameter was defined as (longest diameter+shortest diameter)/2.

Culture of Human Pulmonary Arterial Smooth Muscle Cells

Human PASMCs were obtained from commercial sources (Lonza). Cells were grown in SMC growth medium (5% FBS, 1 μg/ml hydrocortisone, 10 ng/ml human epidermal growth factor, 3 ng/ml basic fibroblast growth factor, 10 μg/ml heparin, 10 μg/ml gentamycin, and 0.25 μg/ml amphotericin) (Lonza) and were used between passages 4 and 7. Cells were starved in SMC medium with 0.1% FBS for 24 h before the experiment. Human PASMCs were cultured in the TGF-β 2.5 ng/ml condition for 6 hours to determine the effect of lumikine on the protein expressions.

Hypoxia Treatment

Human PASMCs were cultured in the hypoxic condition for 24 hours to determine the effect of lumikine on the protein expressions. Hypoxia was created in an incubator: 5% CO2+94% N2+10% O2, 37° C. Lumikine were dissolved in culture medium at a final concentration of 100 nM.

Isolation and Culture of Rat PASMCs

PASMCs were isolated from lungs of MCT-treated or control rats using the explant method, as described previously (Lai Y J et al. Am J Respir Crit Care Med. 2008, 178(2):188-96). To culture PASMCs, the PA was dissected from lung and cardiac tissues. The pulmonary arterial tissue was cut into small pieces and suspended in Dulbecco's modified Eagle's medium (DMEM) (Gibco) containing 100 U/mL penicillin, 100 g/mL streptomycin (Gibco), and 20% fetal bovine serum (FBS) (Gibco). We observed a decrease in desmin, a smooth muscle cell gene marker, at passage 3. Therefore, rat PASMCs were used before passage 3 for all of the in vitro experiments. Characterization of PASMCs was performed by immunocytochemical staining with anti-α-SM-actin (Sigma-Aldrich) and anti-desmin (NeoMakers) antibodies.

Western Blotting.

Tissue samples or cell samples were homogenized in lysis buffer containing 50 mM Tris-HCl, pH 7.4, 50 mM NaCl, 5 mM EDTA, 1% Triton X-100, 0.05% SDS, 50 mM NaF, 10 mM β-glycerophosphate, sodium pyrophosphate, 100 μM Na₃VO₄, and protease inhibitor cocktail (Calbiochem, Millipore). Immunoblotting was performed using anti-lumican (Abcam; 1:2000), ant-HIF-1α(Abeam; 1:1000), anti-p-AKT, or AKT (Cell Signaling; 1:1000), and anti-p-SMAD2 (Cell Signaling; 1:1000), as primary antibodies. Secondary antibodies specific for peroxidase-conjugated anti-mouse IgG (Jackson; 1:10000) or anti-rabbit IgG (Jackson; 1:10000) were used as needed. Blots were visualized using an enhanced chemi-luminescence detection system (Millipore). Samples were normalized to GAPDH (Santa Cruz; 1:10000) and quantified by densitometry.

Immunocytochemical Analysis

In PASMCs was performed with primary antibodies against lumican (Abeam; 1:100). At the end of experiments, cells were rinsed with PBS, fixed with cold methanol for 5 minutes at room temperature; after removal of methanol; washed twice with 1×PBS, blocked with 1% goat serum/1% BSA in PBS for 30 minutes, and incubated with primary antibodies for 1 hour. Following that, cells were incubated with Alexa-488-conjugated (green) (Gibco, Invitrogen) secondary antibodies for lumican for 30 minutes. Nuclei were visualized by DAPI-staining (Gibco, Invitrogen). Fluorescence was observed with a confocal microscope at Microscope Core Laboratory of Chang Gung Memorial Hospital.

Cell Proliferation Assay

The proliferative activity of PASMCs and the effects of the lumikine were determined by an ELISA-based 5-bromo-2-deoxyuridine (BrdU) incorporation assay kit (Roche Diagnostics Co.) according to the manufacturer's instructions.

Data Analysis.

All data are given as mean±SEM. Differences between two groups were determined by unpaired t-test. For multiple groups, one-way ANOVA with post hoc bonferroni's test was used to compare data among groups. A value of P<0.05 was considered to be statistically significant. We also thank American Journal Experts (AJE) for English language editing and the Microscope Core Laboratory, Chang Gung Memorial Hospital, Linkou for technical assistance.

### Example 1 Characterization of Lumican in PAH

1.1 Loss of Lumican was an Indication of PAH

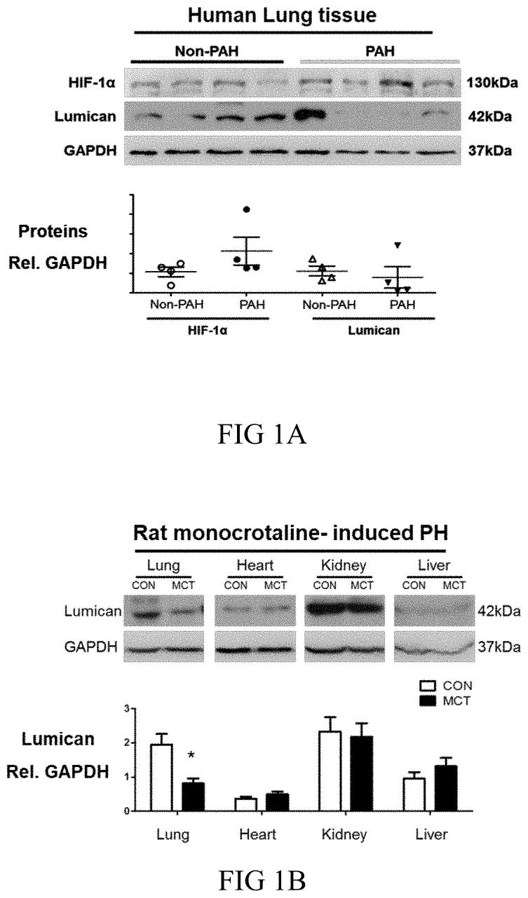

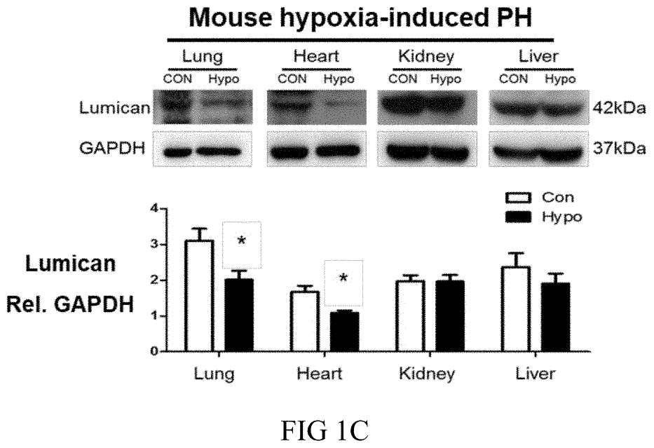

To investigate the expression pattern of lumican in normal and pulmonary hypertensive lungs, lung tissue from 4 IPAH, and 4 individuals without pulmonary hypertension (non-PAH) were examined and compared. In addition, the lung tissues from rats with monocrotaline-induced pulmonary hypertension (MCT-PAH) were also compared to those of the control littermate's rats, and the mice with hypoxia-induced pulmonary hypertension were compared to its control littermates. Results are summarized in FIGS. 1A to 1C.

The HIF1α protein band was detected at 130 kDa. The ratio of the HIF-1α to GAPDH exhibited an increase of the HIF-1α in IPAH lungs, as compared with that in non-PAH, whereas the core protein of lumican was detected at 42 kDa and its level decreased in IPAH lung samples (FIG. 1A).

To characterize whether decrease in lumican level was specific to the lung in rat with pulmonary hypertension, organ-specific levels of lumican protein were compared between control and MCT-PAH rats (FIG. 1B). It was found that decreased lumican level was only observed in the lung of MCT-PAH rats, but not in other organs including kidney, liver and heart (FIG. 1B).

To verify whether decreased lumican level was specific to the lung in mice with hypoxia induced pulmonary hypertension, organ-specific levels of lumican protein were compared between normaxia and hypoxia mice (FIG. 1C). The data confirmed that lumican level was lower in the lung or heart in mice exposed to 10% oxygen for 4 weeks (hypoxia mice), as compared to that of normoxia mice (non-pulmonary hypertensive mice) (FIG. 1C).

Taken together, the results indicated that level of lumican correlated with the severity of PAH in humans and pulmonary hypertension in two murine models of disease. The lung-specific levels of lumican protein decreased in PAH disease.

1.2 the Location of Lumican Expression were Confined to PASMCs in the Lung

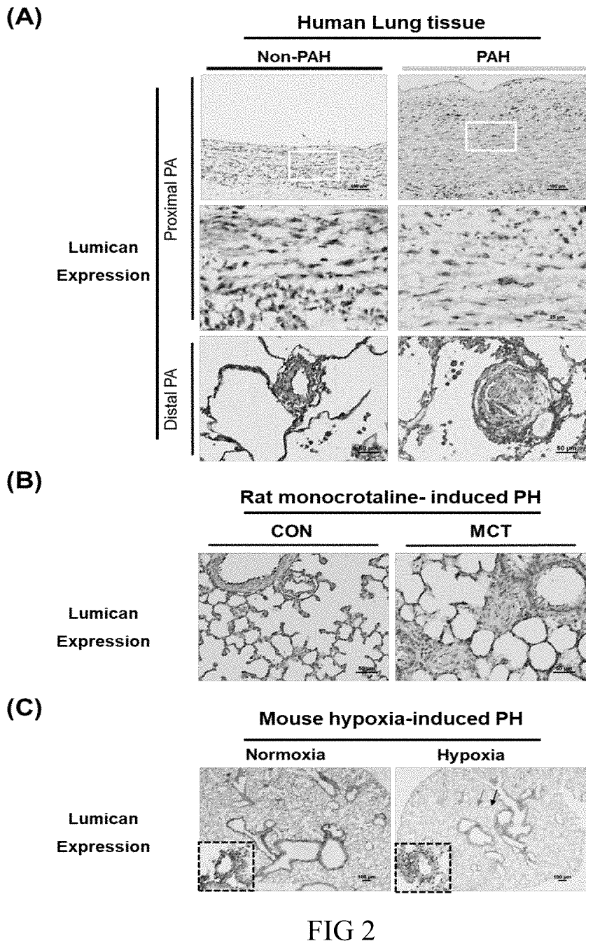

In this example, the locations of lumican expressed in the lung tissues of human and rat were investigated by immunohistochemistry staining. Results are depicted in FIG. 2.

The immunohistochemistry study detected scant expression of lumican in the smooth muscle layer of proximal and distal PAs of patients, as compared to that of the non-PAH patients (FIG. 2, panel A). In MCT-PAH rats, the vascular wall remodeling was recognizable in the muscular pulmonary arteries. The pulmonary artery from the control rat lung section demonstrated lumican-positive staining in the medial smooth muscle wall. The MCT rat lung section exhibited decreased expression of lumican, as compared to that of control rats (FIG. 2, panel B). Similarly, in hypoxia mice, lower level of lumican expression in the distal pulmonary arteries was observed (FIG. 2, panel C). The results indicated that loss of lumican in pulmonary arteries correlated with the severity of PAH.

1.3 Lumican Null Mice Developed Pulmonary Arterial Remodeling and Right Heart Hypertrophy

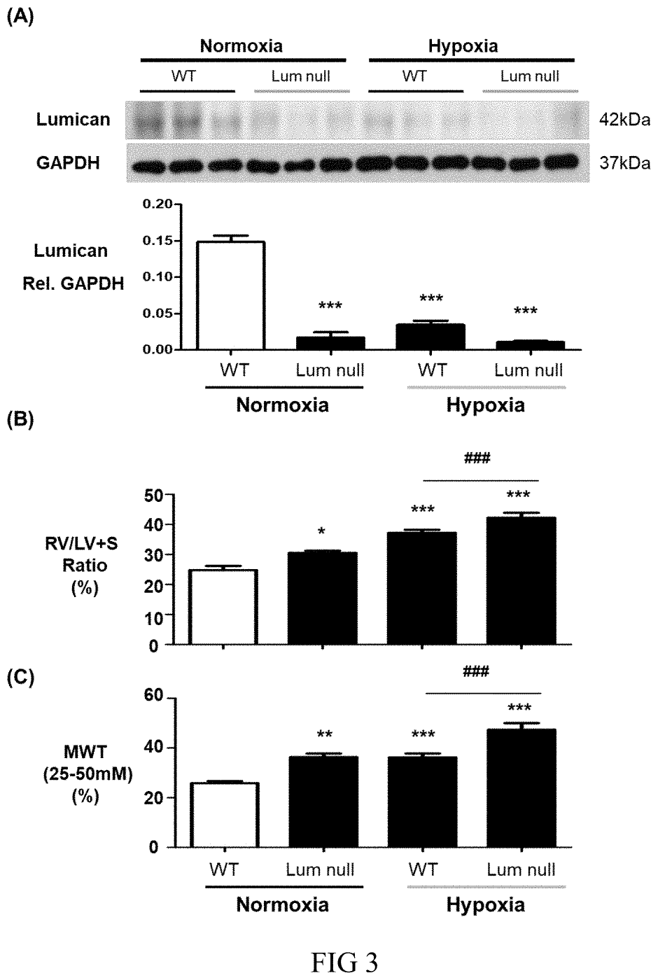

In this example, lumican-null mice generated by targeted gene deletion as described in the “Materials and Methods” section were used to study the development of hypoxia-induced pulmonary hypertension. Results are illustrated in FIG. 3.

It was found that Lumican-null mice had minimal expression of core lumican (42 kDa) in the lung tissue, and the level of lumican decreased when the animals were subjected to hypoxia condition, as compared to that of wild-type mice in the normoxia (FIG. 3, panel A). In addition, the condition of right ventricular hypertrophy and vascular remodeling were found to be worst in lumican-null mice, as compared to those of wild-type mice over a 4-week period of hypoxia (FIG. 3, panels B and C).

In sum, loss of lumican resulted in the development of abnormal right ventricle hypertrophy, and small pulmonary artery luminal narrowing was found to be consistent with advanced pulmonary hypertension.

### Example 2 Characterization of the Present Synthetic Peptide in PAH

2.1 Lumkine Rescued the Hypoxia Induced Reduction in Lumican Expression

In this example, in vitro hypoxic (1% oxygen) model was used to evaluate the effect of the present synthetic peptide—lumikine (SEQ ID NO: 2) on pulmonary arterial smooth muscle cell (PASMCs).

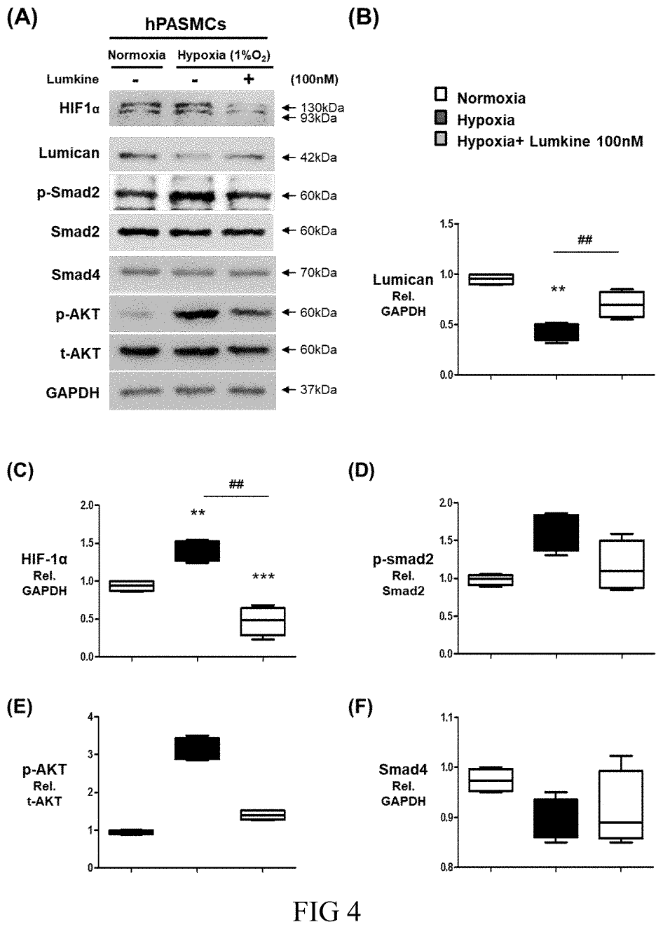

Western blot analysis revealed that human PASMCs subjected to hypoxic condition expressed higher levels of HIF-1α, pSMAD 2/3, and pAKT; and lower level of lumican than those from normoxia PASMC (FIG. 4, panel A). Treatment of PASMCs with lumikine (100 nM) (SEQ ID NO: 2) resulted in an increase in the level of lumican (FIG. 4, panel B), and a reduction in the respective levels of HIF-1α (FIG. 4, panel C), pSmad3 (FIG. 4, panel D), and pAKT (FIG. 4, panel E). Thus, it is reasonably to postulate that lumikine induced lumican expression via inhibiting HIF-1α, p-Smad 3, and pAKT under an in vitro hypoxic condition. Smad4 remained unaffected by lumikine treatment (FIG. 4F).

Together, these data suggest that the lumikine (SEQ ID NO: 2) may rescue hypoxic induced reduction of lumican in PASMCs.

2.2 Lumikine Suppressed the Proliferation of MCT-PAH PASMCs

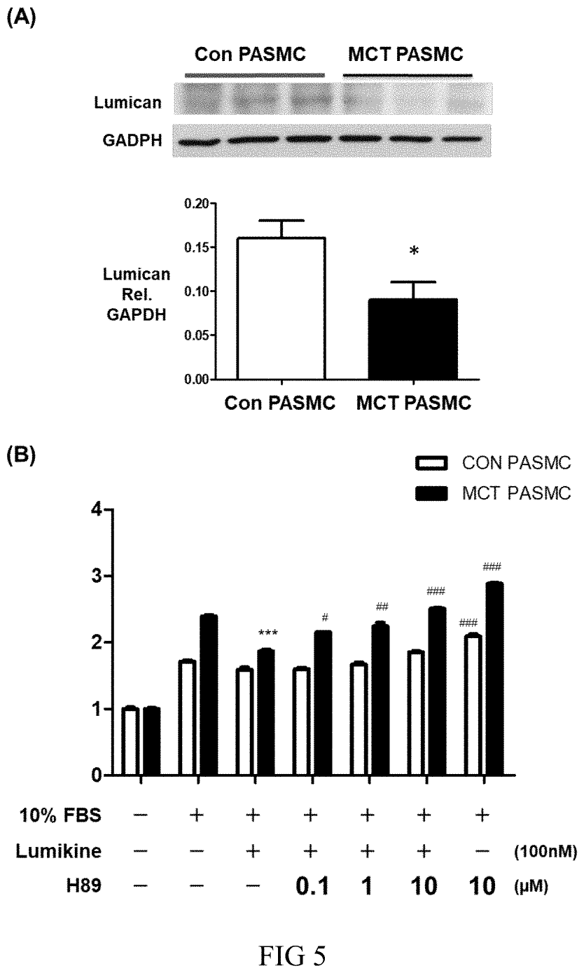

Proliferation of PASMC is critical to the development of PAH. To characterize whether lumikine affected the proliferation of PASMCs in PAH and non-PAH subjects, the PASMCs were isolated from rats with or without MCT induced PAH. As expected, lumican expression was significantly low in MCT PASMC, as compared to control PASMC (FIG. 5, panel A).

Lumican has been demonstrated to inhibit the AKT phosphorylation, which is the crucial factor for cell proliferation, thus, the lumkine suppressed PASMCs proliferation was further studied by use of a PKA inhibitor—H89 that activated p-AKT (10 μM). As expected, the MCT PASMCs with lumikine treatment led to a dose-dependently decrease in serum-induced PASMC proliferation (FIG. 5, panel B), but the effect was reversed by H89 (FIG. 5, panel B). Further, treatment with lumikine had no significantly effects on the proliferation of control PASMCs compared with the values resulting from vehicle treatment (FIG. 5). These data demonstrated that the loss of lumican expression in PASMCs significantly affected cell proliferation and lumikine was capable of inhibiting cell proliferation via activating PKA signaling to inhibit pAKT. Further, treatment with luminkine reduced pAKT expression to suppress the proliferation of MCT PASMCs, but this was reversed by H89, reflecting a crucial role of PKA-dependent pathway in lumkine-induced effects.

Taken together, the data in this example indicated that lumikine may inhibit the proliferation and metabolism of MCT PASMCs by inhibiting the phosphorylation of AKT signaling pathway.

2.3 Lumikine Prevented the Development of Pulmonary Vasculature Remodeling and Right Heart Hypertrophy in Hypoxia-Induced PAH Mice

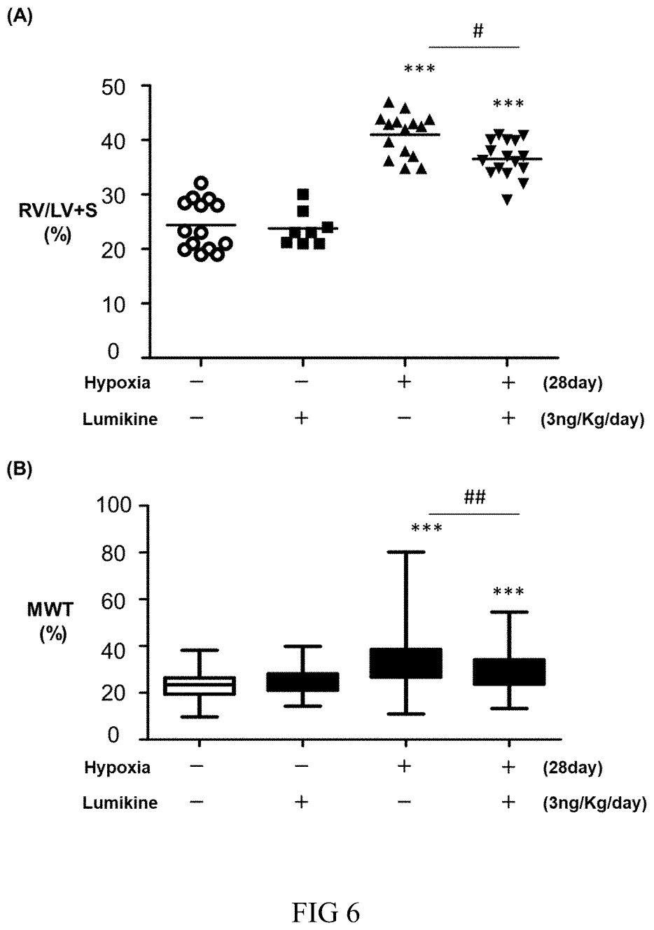

In this example, whether lumikine treatment may prevent the development of PAH in mice was investigated. Mice were induced to develop PAH by subjecting to a hypoxia condition (10% O₂) as described in the “Material and Methods” section. Results are illustrated in FIG. 6.

As depicted, lumikine treatment (3 ng/kg/day, 28 days) significantly reduced right ventricular (RV) weight to the level of the control (FIG. 6, panel A). The degree of PAH was further evaluated by measuring the vascular media wall thickness (MWT), and as expected, the medial wall of pulmonary artery (PA) was significantly thicker in the 28 days hypoxia group, as compared with that in the normoxic animals. Treatment with lumikine (3 ng/kg/day for 21 days) in mice challenged with hypoxia resulted in a significantly reduction the MWT of the PA to the level of the hypoxia group (FIG. 6, panel B).

Taken together, the findings demonstrated that the present synthetic peptide—lumikine (SEQ ID NO: 2), may effectively prevent the development of PAH in mice exposed to hypoxia.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of treating advanced pulmonary arterial hypertension (PAH) in a subject comprising administering to the subject an effective amount of a synthetic peptide of SEQ ID NO: 1 to ameliorate the symptoms associated with the PAH, wherein the SEQ ID NO: 1 is set forth as X₁EX₂LRVANEVTLN (SEQ ID NO: 1), in which X₁ is tyrosine (Y) or phenylalanine (F), and X₂ is alanine (A) or cysteine (C).

2. The method of claim 1, wherein the synthetic peptide has the sequence of SEQ ID NO: 2.

3. The method of claim 1, wherein the synthetic peptide has the sequence of SEQ ID NO: 3.

4. The method of claim 1, wherein the synthetic peptide has the sequence of SEQ ID NO: 4.

5. (canceled)

6. (canceled)

7. The method of claim 1, wherein the synthetic peptide is administered to the subject in the amount of 10 ng/day to 50 mg/day.

8. The method of claim 7, wherein the synthetic peptide is administered to the subject in the amount of 500 ng/day to 30 mg/day.

9. The method of claim 1, further comprising administering to the subject an agent selected from the group consisting of vasodilator, endothelin receptor antagonist, phosphodiesterase 5 (PDE5) inhibitor, calcium channel blocker, soluble guanylate cyclase (SGC) stimulator, anticoagulant, digoxin, diuretics, and oxygen.

10. The method of claim 9, wherein the vasodilator is epoprostenol, iloprost, or treprostinil.

11. The method of claim 9, wherein the endothelin receptor antagonist is bosentan, macitentan, or ambrisentan.

12. The method of claim 9, wherein the PDE5 inhibitor is sildenafil or tadalafil.

13. The method of claim 9, wherein the calcium channel blocker is amlodipine, diltiazem, or nifedipine.

14. The method of claim 9, wherein the SGC stimulator is riociguat.

15. The method of claim 9, wherein the anticoagulant is warfarin.

16. The method of claim 1, wherein the subject is a mammal.

17. The method of claim 16, wherein the subject is a human.


SEQUENCE LISTING (4 sequences)

SEQ ID NO: 1 - protein, 13 aa, Artificial Sequence
  synthetic
  VARIANT 1: X is any amino acid
  VARIANT 3: X is any amino acid
XEXLRVANEVTLN

SEQ ID NO: 2 - protein, 13 aa, Artificial Sequence
  synthetic
YEALRVANEVTLN

SEQ ID NO: 3 - protein, 13 aa, Artificial Sequence
  synthetic
YECLRVANEVTLN

SEQ ID NO: 4 - protein, 13 aa, Artificial Sequence
  synthetic
FEALRVANEVTLN